SEQUENCE LISTING (4 sequences)

SEQ ID NO: 1 - protein, 689 aa, Unknown
  bovine lactoferrin
APRKNVRWCTISQPEWFKCRRWQWRMKKLGAPSITCVRRAFALECIRAIAEKKADAVTLDGGMVFEAGRDPYKLRPVAAEIYGTKESPQTHYYAVAVVKKGSNFQLDQLQGRKSCHTGLGRSAGWIIPMGILRPYLSWTESLEPLQGAVAKFFSASCVPCIDRQAYPNLCQLCKGEGENQCACSSREPYFGYSGAFKCLQDGAGDVAFVKETTVFENLPEKADRDQYELLCLNNSRAPVDAFKECHLAQVPSHAVVARSVDGKEDLIWKLLSKAQEKFGKNKSRSFQLFGSPPGQRDLLFKDSALGFLRIPSKVDSALYLGSRYLTTLKNLRETAEEVKARYTRVVWCAVGPEEQKKCQQWSQQSGQNVTCATASTTDDCIVLVLKGEADALNLDGGYIYTAGKCGLVPVLAENRKSSKHSSLDCVLRPTEGYLAVAVVKKANEGLTWNSLKDKKSCHTAVDRTAGWNIPMGLIVNQTGSCAFDEFFSQSCAPGADPKSRLCALCAGDDQGLDKCVPNSKEKYYGYTGAFRCLAEDVGDVAFVKNDTVWENTNGESTADWAKNLNREDFRLLCLDGTRKPVTEAQSCHLAVAPNHAVVSRSDRAAHVKQVLLHQQALFGKNGKNCPDKFCLFKSETKNLLFNDNTECLAKLGGRPTYEEYLGTEYVTAIANLKKCSTSPLLEACAFLTR

SEQ ID NO: 2 - protein, 7 aa, Artificial Sequence
  Lactoferrin derived peptide
RDPYKLR

SEQ ID NO: 3 - protein, 7 aa, Artificial Sequence
  Lactoferrin derived peptide
KESPQTH

SEQ ID NO: 4 - protein, 7 aa, Artificial Sequence
  Lactoferrin derived peptide
KKCSTSP

(19) United States
(12) Patent Application Publication
(10) Pub. No.: US 2022/0362348 A1
(43) Pub. Date: Nov. 17, 2022

(54) LACTOFERRIN, DERIVED PEPTIDE THEREOF AND METHOD THEREOF FOR INHIBITING AND/OR ALLEVIATING LIPID SYNTHESIS

(71) Applicant: RENORIGIN INNOVATION INSTITUTE CO., LTD., Taipei City (TW)

(72) Inventors: Hsiu-Chin HUANG, Taipei City (TW); Hsuan LIN, Taipei City (TW)

(73) Assignee: RENORIGIN INNOVATION INSTITUTE CO., LTD., Taipei City (TW)

(21) Appl. No.: 17/406,779

(22) Filed: Aug. 19, 2021

(30) Foreign Application Priority Data
May 11, 2021 (TW) 110116995

(51) Int. Cl. A61K 38/40

(52) U.S. Cl. CPC A61K 38/40

(57) ABSTRACT

The present invention provides a lactoferrin, a derived peptide thereof, a composition comprising the same and a use thereof for inhibiting and/or alleviating lipid synthesis. The lactoferrin comprises the amino acid sequence of SEQ ID NO: 01, and the derived peptide of the lactoferrin comprises at least one selected from the amino acid sequences of SEQ ID NO: 02, SEQ ID NO: 03, and SEQ ID NO: 04.

### REFERENCE TO SEQUENCE LISTING SUBMITTED VIA EFS-WEB

This application includes an electronically submitted sequence listing in .txt format. The .txt file contains a sequence listing entitled “5025-0392PUS1_ST25.txt” created Oct. 17, 2021 and is 6,882 bytes in size. The sequence listing contained in this .txt file is part of the specification and is hereby incorporated by reference herein in its entirety.

### BACKGROUND OF THE INVENTION

### Field of the Invention

The present invention relates to a lactoferrin, a derived peptide thereof, a composition comprising the same and a use thereof and, in particular, to a lactoferrin, a derived peptide thereof, a composition comprising the same and a use thereof for inhibiting or alleviating lipid synthesis.

### Description of the Prior Art

The secretion amount of scalp oil (sebum) is regulated by genes and is related to heredity. When the scalp is aging and the function of sebaceous glands in regulating oil secretion is weakened, the sebaceous glands are susceptible to interference from the organism itself and the external environment, and cannot properly regulate oil secretion. Excessive oil secreted by the sebaceous glands will interact with microorganisms and dust on the surface of the scalp, causing inflammation and even folliculitis and seborrheic dermatitis.

In order to solve the problem of excessive oil secretion by the sebaceous glands, the general public usually uses the shampoo containing the surfactant.

The surfactant emulsifies the oily dirt on the hair and scalp, allowing the dirt to enter the water and be easily washed away. The scalp will remain clean and free of oily dirt until new sebum is formed (YangJ (2017) Hair Care Cosmetics. In: Cosmetic Science and Technology: Theoretical Principles and Applications. Elsevier Inc., pp 601-615).

The surfactants or other additives among the main ingredients contained in cleaning products, such as soaps, shampoos, and cleansers, are known to cause dermatitis or skin allergies in users. Among them, sodium dodecyl sulfate (SDS) is an anionic surfactant, has high detergency, and is widely used in cleaning products and researches on irritant contact dermatitis (InamiY, UtaD, AndohT (2020) Neuronal hyperexcitability and astrocyte activation in spinal dorsal horn of a dermatitis mouse model with cutaneous hypersensitivity. Neurosci Lett 720:134784.).

If a shampoo with a strong degreasing effect is used to clean the oily dirt on the hair and scalp, the cells will think that the scalp's oil secretion is insufficient, and instead secrete more oil to protect the scalp. If the more secreted oil is washed out again, the cell will think that the oil is still insufficient and secrete more oil. After several cycles, the cell's self-regulation mechanism may collapse, causing maladjustment of sebum balance.

### SUMMARY OF THE INVENTION

The present invention found that lactoferrin (LF) and derived peptides of lactoferrin can effectively inhibit and/or alleviate the oil synthesis of sebocytes, and can be used as an alternative solution to problems such as improving scalp oil.

In one aspect, the present invention provides a lactoferrin (LF), a derived peptide of the lactoferrin or a combination thereof for inhibiting and/or alleviating lipid synthesis, wherein the lactoferrin comprises the amino acid sequence of SEQ ID NO: 01, and the derived peptide of the lactoferrin comprises at least one selected from the amino acid sequences of SEQ ID NO: 02, SEQ ID NO: 03, and SEQ ID NO: 04.

Preferably, the inhibiting and/or alleviating lipid synthesis refers the lipid synthesis of a subject's cells is inhibited or alleviated so that a content of a synthesized lipid is reduced.

Preferably, a reduction ratio of the content of the synthesized lipid to that of a non-administrated subject ranges from 10 to 40%.

In another aspect, the present invention provides a composition for inhibiting and/or alleviating lipid synthesis, comprising a lactoferrin, a derived peptide of the lactoferrin or a combination thereof, wherein the lactoferrin comprises the amino acid sequence of SEQ ID NO: 01, and the derived peptide of the lactoferrin comprises at least one selected from the amino acid sequences of SEQ ID NO: 02, SEQ ID NO: 03, and SEQ ID NO: 04.

Preferably, the composition comprises about 5 μg/mL to 1000 μg/mL the lactoferrin, the derived peptide of the lactoferrin or the combination thereof.

Preferably, the composition comprises one or more pharmaceutically acceptable carriers.

Preferably, a dosage form of the composition is an ointment, a gel, a cream, an emulsion, a liquid, a wax, a powder, a spray, a gel spray, a foam, a shampoo, a treatment agent, a scalp treatment agent, a tonic, a drop or a patch.

Preferably, the inhibiting and/or alleviating lipid synthesis refers the lipid synthesis of a subject's cells is inhibited or alleviated so that a content of a synthesized lipid is reduced.

Preferably, a reduction ratio of the content of the synthesized lipid to that of a non-administrated subject ranges from 10 to 40%.

In a further aspect, the present invention provides a method for inhibiting and/or alleviating lipid synthesis, comprising administering the composition as described above to a subject.

Preferably, the inhibiting and/or alleviating lipid synthesis is achieved by administering the composition comprising the lactoferrin, the derived peptide of the lactoferrin or the combination thereof to a subject.

Preferably, an effective amount of the composition administered to the subject ranges from 5 μg/mL to 1000 μg/mL.

Preferably, a way to administer the composition to the subject is transdermal.

Preferably, a frequency of administering the composition to the subject is daily.

Preferably, the subject's cells refer to sebocytes.

Preferably, the lipid synthesis is induced by linoleic acid.

Preferably, the inhibiting and/or alleviating lipid synthesis refers the lipid synthesis of the subject's cells is inhibited or alleviated so that a content of a synthesized lipid is reduced.

Preferably, a reduction ratio of the content of the synthesized lipid to that of a non-administrated subject ranges from 10 to 40%.

### BRIEF DESCRIPTION OF THE DRAWINGS

FIG. 1 shows the lipid content of sebocytes in the negative control group, the positive control group, and under different concentrations of lactoferrin. Among them, A is the negative control group, B is the positive control group, and C is lactoferrin for image by stain with Nile red under the fluorescent microscope. D is a statistical bar graph showing the fluorescence intensity of each group, in which * shows a significant difference from the negative control group (control), and # shows a significant difference from the positive control group (LA). The experimental results are shown as the average±standard deviation of three experiments. ***p<0.001.### p<0.001

FIG. 2 shows the lipid content of sebocytes in the negative control group, the positive control group, and under different concentrations of lactoferrin peptide 69-75. Among them, A is the negative control group, B is the positive control group, and C is lactoferrin peptide 69-75 for image by stain with Nile red under the fluorescent microscope. D is a statistical bar graph showing the fluorescence intensity of each group, in which * shows a significant difference from the negative control group (control), and # shows a significant difference from the positive control group (LA). The experimental results are shown as the average±standard deviation of three experiments. ***p<0.001.### p<0.001

FIG. 3 shows the lipid content of sebocytes in the negative control group, the positive control group, and under different concentrations of lactoferrin peptide 85-91. Among them, A is the negative control group, B is the positive control group, and C is lactoferrin peptide 85-91 for image by stain with Nile red under the fluorescent microscope. D is a statistical bar graph showing the fluorescence intensity of each group, in which * shows a significant difference from the negative control group (control), and # shows a significant difference from the positive control group (LA). The experimental results are shown as the average ±standard deviation of three experiments. ***p<0.001.### p<0.001

FIG. 4 shows the lipid content of sebocytes in the negative control group, the positive control group, and under different concentrations of lactoferrin peptide 695-701. Among them, A is the negative control group, B is the positive control group, and C is lactoferrin peptide 695-701 for image by stain with Nile red under the fluorescent microscope. D is a statistical bar graph showing the fluorescence intensity of each group, in which * shows a significant difference from the negative control group (control), and # shows a significant difference from the positive control group (LA). The experimental results are shown as the average±standard deviation of three experiments. ***p<0.001.### p<0.001

FIG. 5 shows the lipid content of sebocytes in the negative control group, the positive control group, and under different kinds of lactoferrin peptides. Among them, A is the negative control group, B is the positive control group, and C, D, E, F, G and H are lactoferrin peptide 69-75, 85-91, 695-701, 137-146, 278-287 and 291-297 for images by stain with Nile red under the fluorescent microscope. I is a statistical bar graph showing the fluorescence intensity of each group, in which * shows a significant difference from the positive control group (LA). The experimental results are shown as the average±standard deviation of three experiments. ***p<0.001.### p<0.001

### DETAILED DESCRIPTION OF THE EMBODIMENTS

Unless otherwise defined, all technical and scientific terms used herein have the same meaning as those understood by people having ordinary skill in the art of the present invention.

When the term “a” or “one” is used together with the term “including” in the claims and/or specification, it means “one”, but it also has the same meaning as “one or more”, “at least one” and “one or more than one”.

The term “peptide” is used herein with its conventional meaning, which means it is a polymer whose monomers are amino acids and are linked to each other by amide bonds. Alternatively, it refers to a polypeptide. When the amino acid is an a-amino acid, L-optical isomer or D-optical isomer can be used. In addition, it may also contain unnatural amino acids such as β-alanine, phenylglycine and homoarginine. The standard abbreviation of amino acids is used.

The term “carrier” as used herein refers to a material that is generally used to prepare pharmaceutical or cosmetic compositions to improve stability, sterility, and delivery. When the peptide delivery system is formulated as a solution or suspension, the delivery system is in an acceptable carrier.

If the sebaceous glands of an individual's scalp secrete too much oil, it will interact with microorganisms and dust on the surface of the scalp, thereby causing an inflammatory reaction, and even folliculitis and seborrheic dermatitis, and more severe cases may cause hair loss. In order to avoid or improve the problems caused by excessive oil, shampoos with a strong degreasing effect are generally used to clean the oily dirt on the scalp or hair. However, the surfactants in shampoos with a strong degreasing effect may cause dermatitis or skin irritation. If shampoos' degreasing effect is too strong, it may also cause cells to secrete more oil due to cells' misjudgement of insufficient secretion of scalp oil, which will eventually lead to the breakdown of cells' self-regulation mechanism in oil secretion and the maladjustment of sebum balance.

In this regard, an object of the present invention is to develop a novel composition, which does not cause discomfort to the user, such as inflammation, or other diseases or symptoms derived therefrom. This novel composition can also start from the source of the problem to inhibit the production of sebum, instead of removing the oil that has been produced, which may cause more serious scalp problems.

In view of the above, the present invention develops a lactoferrin (LF), a derived peptide of the lactoferrin or a combination thereof, which can inhibit the sebaceous glands of the scalp from secreting oil and/or alleviating the oil secretion rate of the sebaceous glands of the scalp. The lactoferrin can be obtained by chemical synthesis or extraction from natural milk. In one embodiment, the lactoferrin is bovine lactoferrin. In a preferred embodiment, the lactoferrin comprises the amino acid sequence of SEQ ID NO: 01. The derived peptide of the lactoferrin can be synthesized chemically. In one embodiment, the derived peptide of the lactoferrin is at least one selected from lactoferrin peptide 69-75, lactoferrin peptide 85-91 and lactoferrin peptide 695-701. In a preferred embodiment, the lactoferrin peptide 69-75 comprises the amino acid sequence of SEQ ID NO: 02, the lactoferrin peptide 85-91 comprises the amino acid sequence of SEQ ID NO: 03, and the lactoferrin peptide 695-701 comprises the amino acid sequence of SEQ ID NO:04.

The present invention also develops a composition, which comprises the lactoferrin, the derived peptide of the lactoferrin or the combination thereof as described above. In one embodiment, the composition comprises the lactoferrin, the derived peptide of the lactoferrin or the combination thereof in an amount of about 5 μg/mL to 1000 μg/mL, preferably about 100 μg/mL to 500 μg/mL, and more preferably about 200 μg/mL to 400 μg/mL.

The composition of the present invention includes a carrier for improving stability, sterility, and delivery without affecting the biological activity of the lactoferrin, the derived peptide of the lactoferrin or the combination thereof. In one embodiment, the composition includes one or more pharmaceutically acceptable carriers. In a preferred embodiment, the acceptable carrier is an aqueous carrier. Various aqueous carriers can be used, such as water, buffer, 0.8% saline, 0.3% glycine, hyaluronic acid and the like. In addition, the composition may contain physiologically acceptable auxiliary substances that can approach physiological conditions as required, such as pH adjusting and buffering agents, solution tension adjusting agents, wetting agents, and the like. Specific examples include sodium acetate, sodium lactate, sodium chloride, potassium chloride, calcium chloride, sorbitan monolaurate, triethanolamine oleate, and the like.

The composition of the present invention, without affecting the biological activity of the lactoferrin, the derived peptide of the lactoferrin or the combination thereof, can be formulated using known and widely used techniques. In one embodiment, the dosage form of the composition is an external agent. The external agent is not particularly limited as long as the composition can be applied, sprayed, or adhered to the part to be administered (affected part) of the skin of the individual to be administered. Examples of external agents include ointments, gels, creams, emulsions, liquids, waxes, powders, sprays, gel sprays, foams, shampoos, treatment agents, scalp treatment agents, tonic, drops or patches. In a preferred embodiment, the dosage form of the composition is an ointment, a gel, a cream, an emulsion, a liquid, a spray, a shampoo, a treatment agent, drops or a patch. The above-mentioned external agents can use the lactoferrin, the derived peptide of the lactoferrin or the combination thereof as active ingredients, and then pharmaceutically acceptable bases or various additives as required can be added as needed.

The ointment preparation of the present invention can be either an oily base or a water-soluble base, and both of them can be easily obtained according to known methods. Oily bases, such as vaseline, have little irritation, and are odorless and excellent in skin protection, softening, scab removal, granulation formation and epithelialization promotion. Although vaseline changes its viscosity and consistency due to temperature and thus has different hardness in winter and summer, it is one of the bases with high safety. Vaseline includes yellow petrolatum and white petrolatum with higher purity, both of which can be used. The water-soluble base is a macrogol-based ointment, which has a strong effect of absorbing and removing aqueous secretions. Ointment (vaseline preparation) can be prepared by dissolving an appropriate amount of the lactoferrin, the derived peptide of the lactoferrin or the combination thereof in distilled water or physiological saline to form an aqueous solution and then further mixing it with known or conventional vaseline and stirring according to the known or conventional methods.

The gel agent (suspension base) of the present invention is a water-containing gel, an anhydrous gel, and a low-water content gel composed of a swellable gel-forming material. Furthermore, the gel agent may also be a hydrogel base or a lyogel base, preferably a transparent hydrogel with inorganic or organic polymers as the base. The gel itself is not absorbed by the skin like preparations containing oil or fat. The hydrogel base is a substance that is fat-free, has an ointment-like consistency, and aims to improve the transdermal absorbability of the drug. The lyogel base is a substance obtained by suspending steelyl alcohol and the like in propylene glycol and gelling, and has excellent transdermal absorbability and hygroscopicity. The gel agent can also be used as a gel spray after being filled in a spray container. The gel agent of the present invention can be obtained by uniformly dispersing the lactoferrin, the derived peptide of the lactoferrin or the combination thereof in a hydrophilic gel base. The hydrophilic gel base includes carboxy vinyl polymer, sodium polyacrylate, (vinyl methyl ether/ethyl maleate) copolymer, polymethacrylate, propylene glycol, and the like. The gel agent may be prepared by dissolving an appropriate amount of the lactoferrin, the derived peptide of the lactoferrin or the combination thereof in distilled water or physiological saline to form an aqueous solution and then further mixing it with known or conventional gel bases and stirring according to known or conventional methods.

The cream (emulsion base) of the present invention may be an oil-in-water base (O/W) (vanishing cream) or a water-in-oil base (W/O) (cold cream). The oil-in-water base has less oil-soluble components than water-soluble components, and has the advantage that the whiteness of the cream disappears when it is applied. The cream has good pushing uniformity, good feeling of use on sweaty skin, excellent cosmetic properties and good skin absorption. The water-in-oil base has less water-soluble components than oil-soluble components, and has a cooling effect when it is applied to and pushed evenly on the skin, so it is also called cold cream.

Compared with ointments or creams, the emulsion of the present invention adheres to the hair and is more suitable for use on hair parts and the like. The type of the emulsion can be any of a suspension emulsion base, an emulsive emulsion, and a solution emulsion base.

The liquid of the present invention is obtained by dissolving the lactoferrin, the derived peptide of the lactoferrin or the combination thereof as an active ingredient in alcohol, propylene glycol, polyethylene glycol, water and the like as a base. In a preferred embodiment, the liquid is composed of an aqueous solution formed by dissolving the lactoferrin, the derived peptide of the lactoferrin or the combination thereof in physiological saline. In the aqueous solution, in addition to physiological saline, a small amount of organic bases, such as alcohol, propylene glycol or polyethylene glycol, may also be mixed.

The spray agent of the present invention is a solution of the lactoferrin, the derived peptide of the lactoferrin or the combination thereof sprayed by gas pressure or hand pressure operation. Spray formulations are more convenient than other formulations when used in a wide range.

The foaming agent of the present invention is made by forming a solution of the lactoferrin, the derived peptide of the lactoferrin or the combination thereof and adding a milder amino acid surfactant therein, and is a spray released in a bubble form. From the viewpoint of scalp adhesion, the foaming agent is excellent in effect.

The shampoo of the present invention is a formulation in which the lactoferrin, the derived peptide of the lactoferrin or the combination thereof is mixed or dissolved in the hair washing emulsion, and can be applied at the same time during shampooing. Since the shampoo contains milder amino acid surfactants, it has the effect of allowing the lactoferrin, the derived peptide of the lactoferrin or the combination thereof to penetrate into the scalp. Especially in the case of excessive sebum secretion, shampoo is advantageous because it can remove excess sebum.

The treatment agent of the present invention is a formulation in which the lactoferrin, the derived peptide of the lactoferrin or the combination thereof is mixed or dissolved in the treatment agent used during hair washing and can be applied at the same time during hair caring. The treatment agent can simultaneously replenish moisture and oil to the scalp when it is applied. The treatment agent is advantageous because it has the functions of moisturizing and replenishing oil to the scalp when being applied.

The scalp treatment agent of the present invention refers to a treatment agent specially formulated with ingredients for moisturizing the scalp and replenishing oil. Most of the scalp treatment agents are blended with various plant extracts, such as rosemary extract, soapberry extract or coconut extract.

The tonic of the present invention can be prepared by formulating the lactoferrin, the derived peptide of the lactoferrin or the combination thereof, and simultaneously formulating the following base materials: 50% to 70% alcohol and water; hinokitiol, panthenol (provitamin B5) or Swertia Japonica extract and other ingredients that have the function of keeping the hair and scalp healthy; dipotassium glycyrrhizinate, which has a female hormone-like action and is formulated with a fungicide, a moisturizing ingredients such as glycerin, salicylic acid that makes dandruff easy to remove, menthol that prevents itching and gives a cooling sensation, fragrance, etc. Tonics are used to prevent dandruff, odor and keep hair clean, while preventing itching, stuffiness and dampness, and eliminating unpleasant symptoms about hair. They are generally used after shampooing.

The composition of the present invention can be applied to the above-mentioned various dosage forms, and other commonly used ingredients, such as thickeners, vitamins, amino acids, anti-wrinkle agents, seaweed extracts, cell activators, transdermal absorption promoters, foaming agents, solubilizers, keratolytic agents, hormones, pigments, plasticizers, inorganic powders, organic powders, etc., can also be appropriately added to these dosage forms according to needs.

According to the above-mentioned lactoferrin, the derived peptide of the lactoferrin or the combination thereof, and the composition containing the same, the present invention further develops a use and method for inhibiting and/or alleviating lipid synthesis. The composition of the present invention achieves the effect of inhibiting and/or alleviating lipid synthesis of the subject's cells by administering the composition comprising the lactoferrin, the derived peptide of the lactoferrin or the combination thereof to the subject. In one embodiment, the effective amount of the composition administered to the subject ranges from 5 μg/mL to 1000 μg/mL, preferably 100 μg/mL to 500 μg/mL, and more preferably 200 μg/mL to 400 μg/mL.

The route of the present invention to administer the composition to the subject depends on the type of the composition and the dosage suitable for the subject. In a preferred embodiment, the route of administration is transdermal, for example. The frequency of the present invention to administer the composition to the subject depends on the type of the composition and the dose suitable for the subject. In one embodiment, the frequency of administration is, for example, daily or weekly.

The composition of the present invention has the effect of inhibiting and/or alleviating lipid synthesis by containing the lactoferrin, the derived peptide of the lactoferrin or the combination thereof. In one embodiment, the inhibition of lipid synthesis refers the lipid synthesis of the cells of the individual (the subject) receiving the administration of the composition of the present invention is inhibited such that the content of lipid synthesized by the cells of the subject after the administration of the composition is reduced compared to that before the administration of the composition. In one embodiment, the alleviation of lipid synthesis refers the lipid synthesis of the cells of the individual (the subject) receiving the administration of the composition of the present invention is alleviated such that the content of lipid synthesized by the cells of the subject after the administration of the composition is reduced compared to that before the administration of the composition. In one embodiment, the reduced percentage of synthesized lipid content is calculated by the following formula: content of lipid synthesized by the cells after the administration of the composition to the subject/content of lipid synthesized by the cells before the administration of the composition to the subject×100%. In one embodiment, the reduction ratio of the content of the synthesized lipid is 10-40%, preferably 20-30%, and more preferably 30-40% of the content of the lipid synthesized by the subject without administration of the composition.

The subject to whom the composition of the present invention is administered is a human or an animal, preferably a human. The cells of the subject of the present invention refer to sebocytes. The mechanism of lipid synthesis targeted by the composition of the present invention is not particularly limited. In one embodiment, in the lipid synthesis mechanism targeted by the composition of the present invention, the lipid synthesis is induced by linoleic acid.

The following examples illustrate the experiments conducted in the research and development of the present invention to further clarify the features and advantages of the present invention. It should be understood that the embodiments are only illustrative examples of the claimed invention, and should not be used to limit the scope of the claims of the present invention.

Materials and Methods

1. Sample Preparation

Bovine lactoferrin was purchased from New Bellus Enterprise Co., Ltd (Tainan, Taiwan) and stored at room temperature for later use. If used for a long time, it should be stored in cold storage. Lactoferrin peptide 69-75, lactoferrin peptide 85-91, and lactoferrin peptide 695-701 were purchased from DgPeptides Co., Ltd (Hangzhou, China). The purity and composition of these lactoferrin peptides were confirmed by high performance liquid chromatography (HPLC) and mass spectrometer. 10 mg of the freeze-dried powder of the peptide was dissolved in 1 mL of double deionized water (ddH₂O) and stored at −20° C. for use as a sample of lactoferrin peptide. Nile red was purchased from Sigma-Aldrich Corporation (Missouri, USA). Nile Red was dissolved in 1 mg/mL acetone and used as a dye for later use.

2. Cell Culture

Human sebocytes (Cat. No. 36079-01, Celprogen, Torrance, Calif., USA) were cultured in Dulbecco's Modified Eagle Medium (DMEM): Nutrient Mixture F-12 (DMEM/F12) purchased from Thermo Fisher Scientific Limited (Barrington, Ill., USA), 10% fetal bovine serum (FBS) purchased from Gibco, Grand Island, N.Y., USA and penicillin/streptomycin (100 IU/50 g/mL), incubated at 37° C. in the 5% CO₂ atmosphere.

3. Neutral Fat Detection

Human sebocytes were implanted in 24- or 96-well plates. After 24 hours of culture, the cells were divided into a negative control group, a positive control group and an experimental group. The negative control group did not receive any treatment, the positive control group was added 0.1 mM linoleic acid (LA), and the experimental group was added 0.1 mM linoleic acid (LA) and 100 to 400 μg/mL bovine lactoferrin, 25 to 100 μg/mL lactoferrin peptide 69-75, lactoferrin peptide 85-91 or lactoferrin peptide 695-701. The treated human sebocytes were cultured for two days. Human sebocytes were fixed with 4% formaldehyde at room temperature. After 60 minutes of fixation, the cells were washed with PBS and stained with Nile Red solution (1 μg/ml in PBS) at 37° C. for 15 minutes. Next, the cells were washed and observed under a fluorescent microscope. Fluorescence intensity is measured with fluorometer Spectra Max i3.

Experimental Results

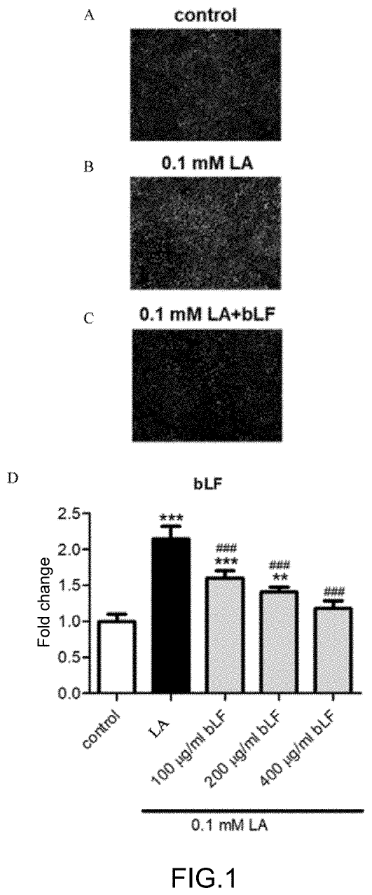

FIG. 1 shows the lipid contents of sebocytes in the negative control group, the positive control group, and the experimental groups with 100 μg/mL, 200 μg/mL and 400 μg/mL lactoferrin respectively. Compared with the negative control group in A, the fluorescence intensity of the positive control group in B increases, indicating that the addition of LA in the positive control group do induce lipid synthesis. The fluorescence intensity of the experimental group in C with 200 μg/mL lactoferrin is reduced compared with that of the positive control group in B, indicating that the addition of lactoferrin in the experimental group can inhibit and/or alleviate the synthesis of lipids induced by LA and reduces the final lipid content. In D, the fluorescence intensity of the negative control group is about 1.0, the fluorescence intensity of the positive control group is about 2.0, the fluorescence intensity of the experimental group with 100 μg/mL lactoferrin is about 1.5, the fluorescence intensity of the experimental group with 200 μg/mL lactoferrin is about 1.5, and the fluorescence intensity of the experimental group with 400 μg/mL lactoferrin is about 1.5. Compared with the fluorescent intensity of the negative control group, the fluorescence intensity of the positive control group shows ***, indicating that there is a significant difference in the content of lipid synthesized by the induction of added LA, and the lipid content is more than 2 times. Compared with the fluorescent intensity of the positive control group, the fluorescence intensity of the experimental group shows ###, indicating that the addition of lactoferrin can inhibit and/or alleviate the content of lipid synthesized by the induction of LA, there is a significant difference in the lipid contents, and the lipid content is reduced by 25% . In addition, as the concentration of the added lactoferrin increases, the lipid content decreases, indicating that the content of lipid synthesized by the induction of LA is inhibited and/or alleviated by lactoferrin in a dose-dependent manner. What's more, the fluorescence intensity of the experimental group with 400 μg/mL lactoferrin is not shown* compared to the fluorescence intensity of the negative control group, indicating that adding lactoferrin to a specific concentration can inhibit and/or alleviate the content of lipid synthesized by the induction of LA to the level that no LA is added and thus no lipid is synthesized.

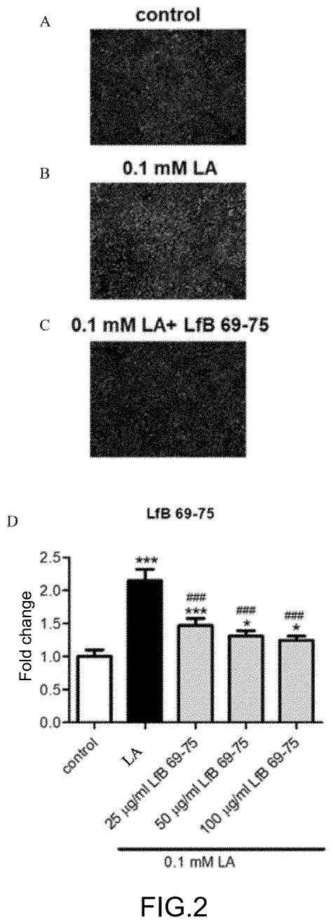

FIG. 2 shows the lipid contents of sebocytes in the negative control group, the positive control group and the experimental groups with 25 μg/mL, 50 μg/mL and 100 μg/mL lactoferrin peptide 69-75 respectively. Compared with the negative control group in A, the fluorescence intensity of the positive control group in B increases, indicating that the addition of LA in the positive control group do induce lipid synthesis. The fluorescence intensity of the experimental group in C with 50 μg/mL lactoferrin peptide 69-75 is reduced compared with that of the positive control group in B, indicating that the addition of lactoferrin peptide 69-75 in the experimental group can inhibit and/or alleviate the synthesis of lipids induced by LA and reduces the final lipid content. In D, the fluorescence intensity of the negative control group is about 1.0, the fluorescence intensity of the positive control group is about 2.0, the fluorescence intensity of the experimental group with 25 μg/mL lactoferrin peptide 69-75 is about 1.5, the fluorescence intensity of the experimental group with 50 μg/mL lactoferrin peptide 69-75 is about 1.5, and the fluorescence intensity of the experimental group with 100 μg/mL lactoferrin peptide 69-75 is about 1.5. Compared with the fluorescent intensity of the negative control group, the fluorescence intensity of the positive control group shows ***, indicating that there is a significant difference in the content of lipid synthesized by the induction of added LA, and the lipid content is more than 2 times. Compared with the fluorescent intensity of the positive control group, the fluorescence intensity of the experimental group shows ###, indicating that the addition of lactoferrin peptide 69-75 can inhibit and/or alleviate the content of lipid synthesized by the induction of LA, there is a significant difference in the lipid contents, and the lipid content is reduced by 25% . In addition, as the concentration of the added lactoferrin peptide 69-75 increases, the lipid content decreases, indicating that the content of lipid synthesized by the induction of LA is inhibited and/or alleviated by lactoferrin peptide 69-75 in a dose-dependent manner.

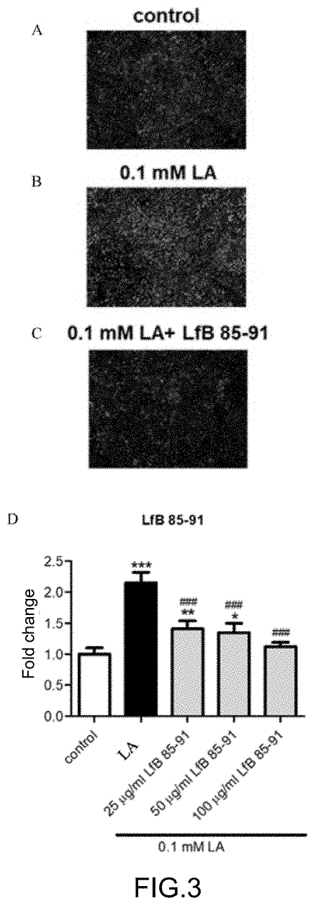

FIG. 3 shows the lipid contents of sebocytes in the negative control group, the positive control group and the experimental groups with 25 μg/mL, 50 μg/mL and 100 μg/mL lactoferrin peptide 85-91 respectively. Compared with the negative control group in A, the fluorescence intensity of the positive control group in B increases, indicating that the addition of LA in the positive control group do induce lipid synthesis. The fluorescence intensity of the experimental group in C with 50 μg/mL lactoferrin peptide 85-91 is reduced compared with that of the positive control group in B, indicating that the addition of lactoferrin peptide 85-91 in the experimental group can inhibit and/or alleviate the synthesis of lipids induced by LA and reduces the final lipid content. In D, the fluorescence intensity of the negative control group is about 1.0, the fluorescence intensity of the positive control group is about 2.0, the fluorescence intensity of the experimental group with 25 μg/mL lactoferrin peptide 85-91 is about 1.5, the fluorescence intensity of the experimental group with 50 μg/mL lactoferrin peptide 85-91 is about 1.5, and the fluorescence intensity of the experimental group with 100 μg/mL lactoferrin peptide 85-91 is about 1.5. Compared with the fluorescent intensity of the negative control group, the fluorescence intensity of the positive control group shows ***, indicating that there is a significant difference in the content of lipid synthesized by the induction of added LA, and the lipid content is more than 2 times. Compared with the fluorescent intensity of the positive control group, the fluorescence intensity of the experimental group shows ###, indicating that the addition of lactoferrin peptide 85-91 can inhibit and/or alleviate the content of lipid synthesized by the induction of LA, there is a significant difference in the lipid contents, and the lipid content is reduced by 25% . In addition, as the concentration of the added lactoferrin peptide 85-91 increases, the lipid content decreases, indicating that the content of lipid synthesized by the induction of LA is inhibited and/or alleviated by lactoferrin peptide 85-91 in a dose-dependent manner. What's more, the fluorescence intensity of the experimental group with 100 μg/mL lactoferrin peptide 85-91 is not shown* compared to the fluorescence intensity of the negative control group, indicating that adding lactoferrin peptide 85-91 to a specific concentration can inhibit and/or alleviate the content of lipid synthesized by the induction of LA to the level that no LA is added and thus no lipid is synthesized.

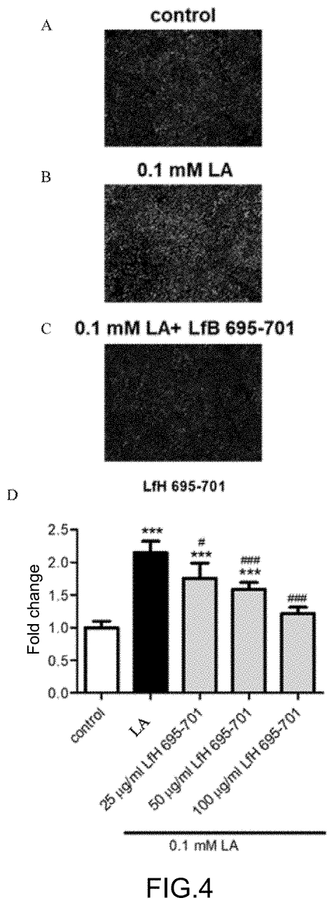

FIG. 4 shows the lipid contents of sebocytes in the negative control group, the positive control group and the experimental groups with 25 μg/mL, 50 μg/mL and 100 μg/mL lactoferrin peptide 695-701 respectively. Compared with the negative control group in A, the fluorescence intensity of the positive control group in B increases, indicating that the addition of LA in the positive control group do induce lipid synthesis. The fluorescence intensity of the experimental group in C with 50 μg/mL lactoferrin peptide 695-701 is reduced compared with that of the positive control group in B, indicating that the addition of lactoferrin peptide 695-701 in the experimental group can inhibit and/or alleviate the synthesis of lipids induced by LA and reduces the final lipid content. In D, the fluorescence intensity of the negative control group is about 1.0, the fluorescence intensity of the positive control group is about 2.0, the fluorescence intensity of the experimental group with 25 μg/mL lactoferrin peptide 695-701 is about 1.5, the fluorescence intensity of the experimental group with 50 μg/mL lactoferrin peptide 695-701 is about 1.5, and the fluorescence intensity of the experimental group with 100 μg/mL lactoferrin peptide 695-701 is about 1.5. Compared with the fluorescent intensity of the negative control group, the fluorescence intensity of the positive control group shows ***, indicating that there is a significant difference in the content of lipid synthesized by the induction of added LA, and the lipid content is more than 2 times. Compared with the fluorescent intensity of the positive control group, the fluorescence intensity of the experimental group shows # or ###, indicating that the addition of lactoferrin peptide 695-701 can inhibit and/or alleviate the content of lipid synthesized by the induction of LA, there is a significant difference in the lipid contents, and the lipid content is reduced by 25% . In addition, as the concentration of the added lactoferrin peptide 695-701 increases, the lipid content decreases, indicating that the content of lipid synthesized by the induction of LA is inhibited and/or alleviated by lactoferrin peptide 695-701 in a dose-dependent manner. What's more, the fluorescence intensity of the experimental group with 100 μg/mL lactoferrin peptide 695-701 is not shown* compared to the fluorescence intensity of the negative control group, indicating that adding lactoferrin peptide 695-701 to a specific concentration can inhibit and/or alleviate the content of lipid synthesized by the induction of LA to the level that no LA is added and thus no lipid is synthesized.

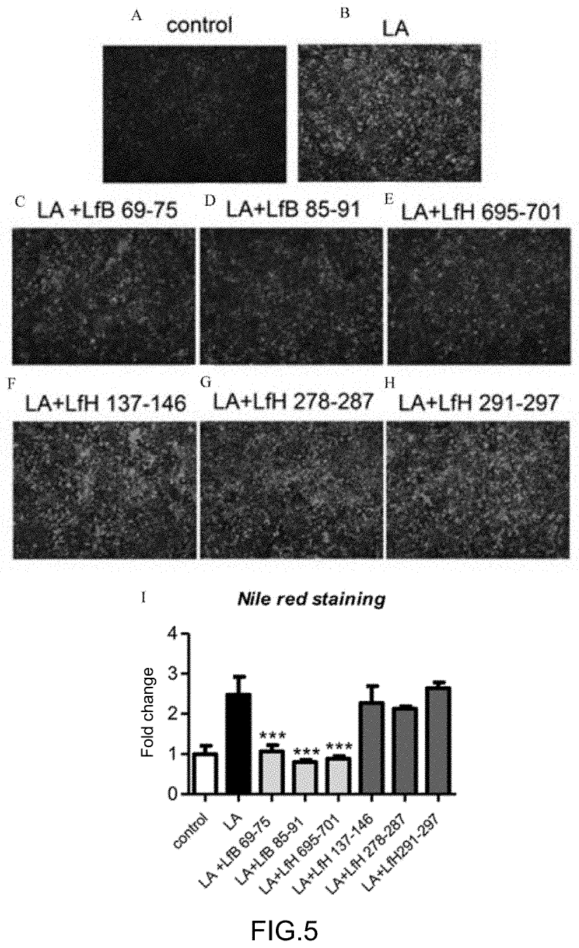

FIG. 5 shows the lipid contents of sebocytes in the negative control group, the positive control group and the experimental groups with 50 μg/mL lactoferrin peptides 69-75, 85-91, 695-701, 137-146, 278-287 and 291-297 respectively. Compared with the negative control group in A, the fluorescence intensity of the positive control group in B increases, indicating that the addition of LA in the positive control group do induce lipid synthesis. The fluorescence intensity of the experimental groups in C, D or E is reduced compared with that of the positive control group in B, indicating that the addition of lactoferrin peptide 69-75, 85-91 or 695-701 in the experimental groups in C, D or E can inhibit and/or alleviate the synthesis of lipids induced by LA and reduce the final lipid content. However, the fluorescence intensity of the experimental group in F, G or H was not significantly reduced compared to that of the positive control group in B, indicating that the addition of lactoferrin 137-146, 278-287 or 291-297 in the experimental group in F, G or H could not significantly inhibit and/or alleviate the synthesis of lipid induced by LA, and thus could not greatly reduce the final lipid content. In D, the fluorescence intensity of the negative control group is about 1.0, the fluorescence intensity of the positive control group is about 2.5, the fluorescence intensity of the experimental group with lactoferrin peptide 69-75, 85-91 or 695-701 is about 1.5, and the fluorescence intensity of the experimental group with lactoferrin peptide 137-146, 278-287 or 291-297 is about 2.0. The fluorescence intensity of the positive control group was more than 2 times that of the negative control group, indicating that the content of lipid synthesized by the induction of added LA was more than 2 times. Compared with the fluorescence intensity of the positive control group, the fluorescence intensity of the experimental group with lactoferrin peptide 69-75, 85-91 or 695-701 shows ***, indicating that the addition of lactoferrin peptide 69-75, 85-91 or 695-701 can inhibit and/or alleviate the content of lipid synthesized by the induction of LA, there is a significant difference in the lipid contents, and the lipid content is reduced by 40%. The fluorescence intensity of the experimental group with lactoferrin peptide 137-146, 278-287 or 291-297 is not shown* compared to the fluorescence intensity of the positive control group, indicating that adding lactoferrin peptide 137-146, 278-287 or 291-297 cannot inhibit and/or alleviate the content of lipid synthesized by the induction of LA to reach a significant difference. It can be seen that not all peptides of lactoferrin are sufficient to exhibit the effect of inhibiting and/or alleviating the content of lipid synthesized by the induction of LA. Screening and experiments are still required to verify the applicability of the peptides of lactoferrin in inhibiting and/or alleviating the synthesis of lipid.

In addition, the experiment of FIG. 5 shows that not all lactoferrin peptides can inhibit and/or alleviate the synthesis of lipids, which can only be confirmed through experiments.

People having ordinary skill in the art will understand that the above specific embodiments can be modified without departing from the broad inventive concept of the present invention. Therefore, it should be understood that the present invention is not limited to the specific embodiments disclosed, but is intended to cover those modifications within the spirit and scope of the present invention as defined by the appended claims.

What is claimed is:

1. A lactoferrin (LF), a derived peptide of the lactoferrin or a combination thereof for inhibiting and/or alleviating lipid synthesis, wherein the lactoferrin comprises the amino acid sequence of SEQ ID NO: 01, and the derived peptide of the lactoferrin comprises at least one selected from the amino acid sequences of SEQ ID NO: 02, SEQ ID NO: 03, and SEQ ID NO: 04.

2. The lactoferrin, the derived peptide of the lactoferrin or the combination thereof of claim 1, wherein the inhibiting and/or alleviating lipid synthesis refers the lipid synthesis of a subject's cells is inhibited or alleviated so that a content of a synthesized lipid is reduced.

3. The lactoferrin, the derived peptide of the lactoferrin or the combination thereof of claim 2, wherein a reduction ratio of the content of the synthesized lipid to that of a non-administrated subject ranges from 10 to 40%.

4. A composition for inhibiting and/or alleviating lipid synthesis, comprising a lactoferrin, a derived peptide of the lactoferrin or a combination thereof, wherein the lactoferrin comprises the amino acid sequence of SEQ ID NO: 01, and the derived peptide of the lactoferrin comprises at least one selected from the amino acid sequences of SEQ ID NO: 02, SEQ ID NO: 03, and SEQ ID NO: 04.

5. The composition of claim 4, wherein the composition comprises about 5 μg/mL to 1000 μg/mL the lactoferrin, the derived peptide of the lactoferrin or the combination thereof.

6. The composition of claim 4, wherein the composition comprises one or more pharmaceutically acceptable carriers.

7. The composition of claim 4, wherein a dosage form of the composition is an ointment, a gel, a cream, an emulsion, a liquid, a wax, a powder, a spray, a gel spray, a foam, a shampoo, a treatment agent, a scalp treatment agent, a tonic, a drop or a patch.

8. The composition of claim 4, wherein the inhibiting and/or alleviating lipid synthesis refers the lipid synthesis of a subject's cells is inhibited or alleviated so that a content of a synthesized lipid is reduced.

9. The composition of claim 8, wherein a reduction ratio of the content of the synthesized lipid to that of a non-administrated subject ranges from 10 to 40%.

10. A method for inhibiting and/or alleviating lipid synthesis, comprising administering the composition of claim 4 to a subject.

11. The method of claim 10, wherein the inhibiting and/or alleviating lipid synthesis is achieved by administering the composition comprising the lactoferrin, the derived peptide of the lactoferrin or the combination thereof to a subject.

12. The method of claim 11, wherein an effective amount of the composition administered to the subject ranges from 5 μg/mL to 1000 μg/mL.

13. The method of claim 11, wherein a route of administering the composition to the subject is transdermal.

14. The method of claim 11, wherein a frequency of administering the composition to the subject is daily.

15. The method of claim 11, wherein the inhibiting and/or alleviating lipid synthesis refers the lipid synthesis of the subject's cells is inhibited or alleviated so that a content of a synthesized lipid is reduced.

16. The method of claim 15, wherein a reduction ratio of the content of the synthesized lipid to that of a non-administrated subject ranges from 10 to 40%.

17. The method of claim 15, wherein the subject's cells refer to sebocytes.

18. The method of claim 10, wherein the lipid synthesis is induced by linoleic acid.
